(19) United States
(12) Patent Application Publication
(10) Pub. No.: US 2022/0125791 A1
(43) Pub. Date: Apr. 28, 2022

(54) NOVEL DEVICES

(71) Applicant: INTRA-CELLULAR THERAPIES, INC., New York, NY (US)

(72) Inventors: Robert DAVIS, San Diego, CA (US); Allen A. FIENBERG, Putnam Valley, NY (US)

(73) Assignee: INTRA-CELLULAR THERAPIES, INC., New York, NY (US)

(21) Appl. No.: 17/426,805

(22) PCT Filed: Jan. 28, 2020
(86) PCT No.: PCT/US20/15474

§ 371 Date: Jul. 29, 2021

Related U.S. Application Data
(60) Provisional application No. 62/798,628, filed on Jan. 30, 2019.

(51) Int. Cl. A61K 31/519; A61K 9/00

(52) U.S. Cl. CPC A61K 31/519; A61K 9/0051

(57) ABSTRACT

Disclosed herein are novel drug eluting contact lenses configured to release one or more compounds that inhibit phosphodiesterase 1 (PDE1). The PDE1 inhibitors may be administered as monotherapy or in combination with additional pharmaceutical agents in the treatment of an ophthalmic disease, disorder or injury. Related methods of use and treatment are also disclosed.

### FIELD OF THE DISCLOSURE

The field relates to devices and compositions comprising inhibitors of phosphodiesterase 1 (PDE1) for treatment of ophthalmic disorders, e.g., topical or systemic treatment of glaucoma or elevated intraocular pressure, and to ophthalmic formulations of cyclic nucleotide Phosphodiesterase 1 (PDE1) inhibitors.

### BACKGROUND

Eleven families of phosphodiesterases (PDEs) have been identified but only PDEs in Family I, the Ca2+-calmodulin-dependent phosphodiesterases (CaM-PDEs), have been shown to mediate the calcium and cyclic nucleotide (e.g. cAMP and cGMP) signaling pathways. The three known CaM-PDE genes, PDE1A, PDE1B, and PDE1C, are all expressed in central nervous system tissue. PDE1A is expressed throughout the brain with higher levels of expression in the CA1 to CA3 layers of the hippocampus and cerebellum and at a low level in the striatum. PDE1A is also expressed in the lung, kidney and heart. PDE1B is predominately expressed in the striatum, pre-frontal cortex, dentate gyrus, olfactory tract and cerebellum, and its expression correlates with brain regions having high levels of dopaminergic innervation. Although PDE1B is primarily expressed in the central nervous system it has been shown to be a mediator of monocyte to macrophage pro-inflammatory transition (Bender and Beavo, 2004, Cell Signaling, 16, 3, p305). PDE1C is primarily expressed in olfactory epithelium, cerebellar granule cells, and striatum. PDE1C is a major phosphodiesterase expressed in the heart and vascular smooth muscle tissue.

Cyclic nucleotide phosphodiesterases decrease intracellular cAMP and cGMP signaling by hydrolyzing these cyclic nucleotides to their respective inactive 5′-monophosphates (5′AMP and 5′GMP). CaM-PDEs play a critical role in mediating signal transduction in brain cells, particularly within an area of the brain known as the basal ganglia or striatum. For example, NMDA-type glutamate receptor activation and/or dopamine D2 receptor activation result in increased intracellular calcium concentrations, leading to activation of effectors such as calmodulin-dependent kinase II (CaMKII) and calcineurin and to activation of CaM-PDEs, resulting in reduced cAMP and cGMP. Dopamine D1 receptor activation, on the other hand, leads to activation of calcium dependent nucleotide cyclases, resulting in increased cAMP and cGMP. These cyclic nucleotides in turn activate protein kinase A (PKA; cAMP-dependent protein kinase) and/or protein kinase G (PKG; cAMP-dependent protein kinase) that phosphorylate downstream signal transduction pathway elements such as DARPP-32 (dopamine and cAMP-regulated phosphoprotein) and cAMP responsive element binding protein (CREB). PDE1B is closely co-localized with the D1 receptor and thought to be a major system to turn off this receptor by hydrolyzing cAMP generated via this receptor.

Glaucoma is an eye disorder characterized by increased intraocular pressure, cupping of the optic disc, and visual field loss. Although the pathophysiological mechanism of open angle glaucoma is still unknown, there is substantial evidence to suggest that increased intraocular pressure is detrimental to the eye, and that the increased intraocular pressure in glaucoma is the most important factor causing degenerative changes in the retina. In one particular form of glaucoma, low tension glaucoma, the actual situation may simply be that the eye is unusually sensitive to pressure and therefore damage may occur at intraocular pressure levels otherwise regarded as physiologically normal.

On the other hand, some individuals may exhibit an abnormally high intraocular pressure substantially without any manifest defects in the visual field or optic disc. Such individuals are referred to as ocular hypertensives. If untreated, glaucoma almost invariably leads to blindness. The course of the disease typically is slow with progressive loss of vision. The basic principle of glaucoma treatment is to lower the intraocular pressure, either by drugs, laser treatment or surgery. The modality of treatment with drugs comprises typically instillation of a prostaglandin, pilocarpine, epinephrine, or topical beta-blocker treatment, e.g. with timolol, as well as systemically administered inhibitors of carbonic anhydrase, e.g. acetazolamide. Cholinesterase inhibitors such as physostigmine and echothiopate may also be employed and have an effect similar to that of pilocarpine. Drugs currently used to treat glaucoma thus include, e.g.,
a. Prostaglandin analogs such as latanoprost (Xalatan), bimatoprost (Lumigan) and travoprost (Travatan), which increase uveoscleral outflow of aqueous humor. Bimatoprost also increases trabecular outflow.
b. Topical beta-adrenergic receptor antagonists such as timolol, levobunolol (Betagan), and betaxolol, which decrease aqueous humor production by the ciliary body.
c. Alpha₂-adrenergic agonists such as brimonidine (Alphagan), which work by a dual mechanism, decreasing aqueous production and increasing uveo-scleral outflow.
d. Less-selective sympathomimetics like epinephrine and dipivefrin (Propine) increase outflow of aqueous humor through trabecular meshwork and possibly through uveoscleral outflow pathway, probably by a beta₂-agonist action.
e. Miotic agents (parasympathomimetics) like pilocarpine work by contraction of the ciliary muscle, tightening the trabecular meshwork and allowing increased outflow of the aqueous humour.
f. Carbonic anhydrase inhibitors like dorzolamide (Trusopt), brinzolamide (Azopt), acetazolamide (Diamox) lower secretion of aqueous humor by inhibiting carbonic anhydrase in the ciliary body.
g. Physostigmine, a cholinesterase inhibitor that is rapidly absorbed through membranes and can be applied topically to the conjunctiva is also used to treat glaucoma.

Although with many of these drugs, the positive effects obtained are at least appreciable, concomitant adverse side-effects are often encountered which tend to diminish the usefulness of the drugs and may negatively affect patient compliance. For example, prostanoids use has been reported to be associated with exacerbation of uveitis and cystoid macular edema. Of some concern is the ability of these agents to cause permanent iris color changes. Blue/green iris color may become brownish. Improvements in these respects are desirable, as well as improvements in drug efficacy. Further, alpha adrenergic agonists use is associated with side effects including conjunctival hyperemia (the eye appears red) along with conjunctival follicle formation. Severe hypotension and other cardiovascular side effects have been reported in infants and toddlers with alpha agonists. Finally, patient adherence to a drug regimen is a known issue in treating most eye disorders.

Use of nonselective PDE inhibitors in combination with IOP lowering agents was suggested some years ago e.g., in EP 0583821 and U.S. Pat. No. 5,079,253. However, nonselective PDE inhibitors may presents risks of side effects and may even interfere with normal ocular function, altering function in photoreceptive cells. PDE6 inhibition, in particular, would clearly lead to loss of vision as cGMP is essential to visual function. For whatever reason, nonselective PDE inhibitors have not been further developed for this purpose. There is a need for methods of treatment that can effectively treat glaucoma without interfering with normal ocular function or presenting unacceptable side effects. There is further need to treat patients in a way that will not interfere with their daily lives while also encouraging adherence to a drug regimen.

### SUMMARY

Provided herein are novel methods and compositions for the treatment or prophylaxis of an opthalmic disease, disorder or injury mediated by PDE1 (e.g., glaucoma or intraocular pressure) that may be ameliorated by administration of a specific inhibitor of phosphodiesterase type I (PDE1 inhibitor). The inventors have surprisingly found that the PDE1 inhibitors of the present application show a high effectiveness in penetrating ocular tissue. Further, many people are already familiar with the use of daily contact lenses, and thus use of a contact lens as a device for drug deposition would enhance patient adherence to a drug regimen to treat such ophthalmic diseases, disorders or injuries. We have shown that PDE1 inhibitors are neuroprotective and stimulate synaptogenesis after neuronal insult.

Thus, in some embodiments, the present disclosure provides for a drug eluting contact lens comprising a pharmaceutically effective amount of a PDE1 inhibitor (e.g., a compound according to Formula I, Ia, II, III, or IV) encapsulated in a drug release matrix for treatment or prophylaxis of an opthalmic disease, disorder or injury mediated by PDE1 (e.g., glaucoma or intraocular pressure). In various aspects of the embodiments, the PDE1 inhibitor is released over an extended period of time.

In some embodiments, the present disclosure provides for a method for treatment or prophylaxis of an opthalmic disease, disorder or injury mediated by PDE1 (e.g., glaucoma or intraocular pressure), the method comprising affixing a drug-eluting contact lens comprising a PDE1 inhibitor encapsulated in a drug release matrix to a patient's eye. In various aspects of the embodiments, the drug-eluting contact lens further comprises one or more pharmaceutically active agents.

In some embodiments, the present disclosure provides for a combination therapy comprising a PDE1 inhibitor (e.g., a compound according to Formula I, Ia, II, III, or IV) and an additional pharmaceutically active agent, wherein both the PDE1 inhibitor and the pharmaceutically active agent are encapsulated in a drug release matrix. In some aspects of the embodiments, the drug release matrix is comprised in a contact lens.

### BRIEF DESCRIPTION OF THE FIGURES

Other aspects, features, benefits and advantages of the embodiments will be apparent with regard to the following description, claims and figures.

FIG. 1 shows the availability of a compound according to the present disclosure for treatment in ocular tissue following topical administration.

### DETAILED DESCRIPTION OF THE DISCLOSURE

In some embodiments, the PDE1 inhibitors for use in the methods of treatment and prophylaxis described herein are selective PDE1 inhibitors.

### PDE1 Inhibitors

In one embodiment the invention provides that the PDE1 inhibitors for use in the methods of treatment and prophylaxis described herein are compounds of Formula I:

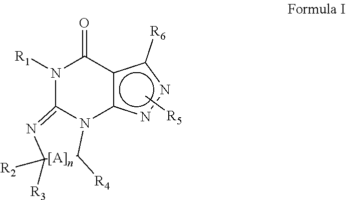

wherein
(i) R₁ is H or C₁₋₄ alkyl (e.g., methyl);
(ii) R₄ is H or C₁₋₄ alkyl and R₂ and R₃ are, independently, H or C₁₋₄ alkyl (e.g., R₂ and R₃ are both methyl, or R₂ is H and R₃ is isopropyl), aryl, heteroaryl, (optionally hetero)arylalkoxy, or (optionally hetero)arylalkyl; or
   R₂ is H and R₃ and R₄ together form a di-, tri- or tetramethylene bridge (pref. wherein the R₃ and R₄ together have the cis configuration, e.g., where the carbons carrying R₃ and R₄ have the R and S configurations, respectively);
(iii) R₅ is a substituted heteroarylalkyl, e.g., substituted with haloalkyl;
   or R₅ is attached to one of the nitrogens on the pyrazolo portion of Formula I and is a moiety of Formula A

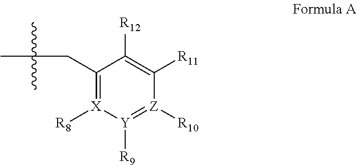

wherein X, Y and Z are, independently, N or C, and R₈, R₉, R₁₁ and R₁₂ are independently H or halogen (e.g., Cl or F), and R₁₀ is halogen, alkyl, cycloalkyl, haloalkyl (e.g., trifluoromethyl), aryl (e.g., phenyl), heteroaryl (e.g., pyridyl (for example pyrid-2-yl) optionally substituted with halogen, or thiadiazolyl (e.g., 1,2,3-thiadiazol-4-yl)), diazolyl, triazolyl, tetrazolyl, arylcarbonyl (e.g., benzoyl), alkylsulfonyl (e.g., methylsulfonyl), heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl (e.g., benzyl), arylamino (e.g., phenylamino), heterarylamino, N,N-dialkylamino, N,N-diarylamino, or N-aryl-N-(arylakyl)amino (e.g., N-phenyl-N-(1,1′-biphen-4-ylmethyl)amino); and
(v) n=0 or 1;
(vi) when n=1, A is —C(R₁₃R₁₄)—
   wherein R₁₃ and R₁₄, are, independently, H or C₁₋₄ alkyl, aryl, heteroaryl, (optionally hetero)arylalkoxy or (optionally hetero)arylalkyl;
      in free, pharmaceutically acceptable salt or prodrug form, including its enantiomers, diastereoisomers and racemates.

In another embodiment the invention provides that the PDE1 inhibitors for use in the methods of treatment and prophylaxis described herein are Formula 1a:

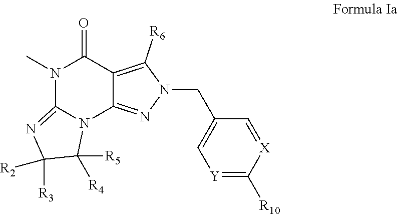

wherein
(i) R₂ and R₅ are independently H or hydroxy and R₃ and R₄ together form a tri- or tetra-methylene bridge [pref. with the carbons carrying R₃ and R₄ having the R and S configuration respectively]; or R₂ and R₃ are each methyl and R₄ and R₅ are each H; or R₂, R₄ and R₅ are H and R₃ is isopropyl [pref. the carbon carrying R₃ having the R configuration];
(ii) R₆ is (optionally halo-substituted) phenylamino, (optionally halo-substituted) benzylamino, C₁₋₄alkyl, or C₁₋₄alkyl sulfide; for example, phenylamino or 4-fluorophenylamino;
(iii) R₁₀ is C₁₋₄alkyl, methylcarbonyl, hydroxyethyl, carboxylic acid, sulfonamide, (optionally halo- or hydroxy-substituted) phenyl, (optionally halo- or hydroxy-substituted) pyridyl (for example 6-fluoropyrid-2-yl), or thiadiazolyl (e.g., 1,2,3-thiadiazol-4-yl); and
(iv) X and Y are independently C or N,
in free, pharmaceutically acceptable salt or prodrug form, including its enantiomers, diastereoisomers and racemates.

In another embodiment the invention provides that the PDE1 inhibitors for use in the methods of treatment and prophylaxis described herein are compounds of Formula II:

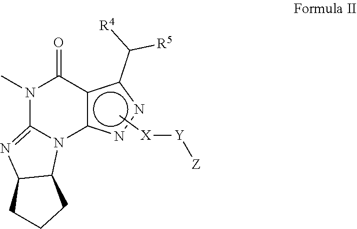

(i) X is C₁₋₆alkylene (e.g., methylene, ethylene or prop-2-yn-1-ylene);
(ii) Y is a single bond, alkynylene (e.g., —C≡C—), arylene (e.g., phenylene) or heteroarylene (e.g., pyridylene);
(iii) Z is H, aryl (e.g., phenyl), heteroaryl (e.g., pyridyl, e.g., pyrid-2-yl), halo (e.g., F, Br, Cl), haloC₁₋₆alkyl (e.g., trifluoromethyl), C(O) R¹, N(R²)(R³), or C₃₋₇cycloalkyl optionally containing at least one atom selected from a group consisting of N or O (e.g., cyclopentyl, cyclohexyl, tetrahydro-2H-pyran-4-yl, or morpholinyl);
(iv) R¹ is C₁₋₆alkyl, haloC₁₋₆alkyl, OH or OC₁₋₆alkyl (e.g., OCH₃);
(v) R² and R³ are independently H or C₁-6alkyl;
(vi) R⁴ and R⁵ are independently H, C₁₋₆alky or aryl (e.g., phenyl) optionally substituted with one or more halo (e.g., fluorophenyl, e.g., 4-fluorophenyl), hydroxy (e.g., hydroxyphenyl, e.g., 4-hydroxyphenyl or 2-hydroxyphenyl) or C₁₋₆alkoxy;
(vii) wherein X, Y and Z are independently and optionally substituted with one or more halo (e.g., F, Cl or Br), C₁₋₆alkyl (e.g., methyl), haloC₁₋₆alkyl (e.g., trifluoromethyl), for example, Z is heteroaryl, e.g., pyridyl substituted with one or more halo (e.g., 6-fluoropyrid-2-yl, 5-fluoropyrid-2-yl, 6-fluoropyrid-2-yl, 3-fluoropyrid-2-yl, 4-fluoropyrid-2-yl, 4,6-dichloropyrid-2-yl), haloC₁₋₆alkyl (e.g., 5-trifluoromethylpyrid-2-yl) or C₁₋₆-alkyl (e.g., 5-methylpyrid-2-yl), or Z is aryl, e.g., phenyl, substituted with one or more halo (e.g., 4-fluorophenyl),

in free, pharmaceutically acceptable salt or prodrug form, including its enantiomers, diastereoisomers and racemates.

In yet another embodiment the invention provides that the PDE1 inhibitors for use in the methods of treatment and prophylaxis described herein are Formula III:

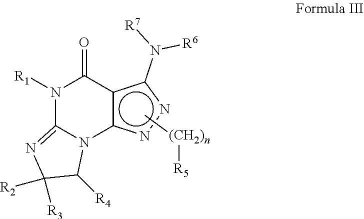

wherein
(i) R₁ is H or C₁₋₄ alkyl (e.g., methyl or ethyl);
(ii) R₂ and R₃ are independently H or C₁₋₆ alkyl (e.g., methyl or ethyl);
(iii) R₄ is H or C₁₋₄ alkyl (e.g., methyl or ethyl);
(iv) R₅ is aryl (e.g., phenyl) optionally substituted with one or more groups independently selected from —C(═O)—C₁₋₆ alkyl (e.g., —C(═O)—CH₃) and C₁₋₆-hydroxyalkyl (e.g., 1-hydroxyethyl);
(v) R₆ and R₇ are independently H or aryl (e.g., phenyl) optionally substituted with one or more groups independently selected from C₁₋₆ alkyl (e.g., methyl or ethyl) and halogen (e.g., F or Cl), for example unsubstituted phenyl or phenyl substituted with one or more halogen (e.g., F) or phenyl substituted with one or more C₁₋₆ alkyl and one or more halogen or phenyl substituted with one C₁₋₆ alkyl and one halogen, for example 4-fluorophenyl or 3,4-difluorophenyl or 4-fluoro-3-methylphenyl; and
(vi) n is 1, 2, 3, or 4,
   in free, pharmaceutically acceptable salt or prodrug form, including its enantiomers, diastereoisomers and racemates.

In yet another embodiment the invention provides that the PDE1 inhibitors for use in the methods of treatment and prophylaxis described herein are Formula IV:

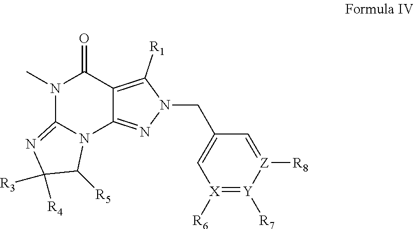

in free or salt form, wherein
(i) R₁ is C₁₋₄alkyl (e.g., methyl or ethyl), or —NH(R₂), wherein R₂ is phenyl optionally substituted with halo (e.g., fluoro), for example, 4-fluorophenyl;
(ii) X, Y and Z are, independently, N or C;
(iii) R₃, R₄ and R₅ are independently H or C₁₋₄alkyl (e.g., methyl); or R₃ is H and R₄ and R₅ together form a tri-methylene bridge (pref. wherein the R₄ and R₅ together have the cis configuration, e.g., where the carbons carrying R₄ and R₅ have the R and S configurations, respectively),
(iv) R₆, R₇ and R₈ are independently:
   H,
   C₁₋₄alkyl (e.g., methyl),
   pyrid-2-yl substituted with hydroxy, or
   —S(O)₂—NH₂;
(v) Provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy,
   in free, pharmaceutically acceptable salt or prodrug form, including its enantiomers, diastereoisomers and racemates.

In one embodiment the invention provides administration of a PDE1 inhibitor for use in the methods of treatment and prophylaxis described herein, wherein the inhibitor is a compound according to the following:

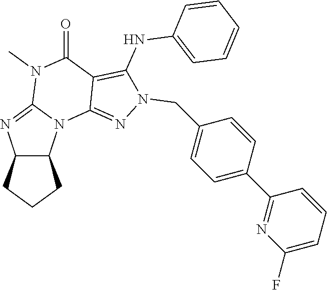 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.

In still another embodiment, the invention provides administration of a PDE1 inhibitor for use in the methods of treatment and prophylaxis described herein, wherein the inhibitor is a compound according to the following:

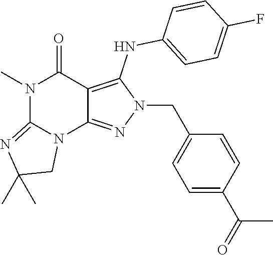 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.

In still another embodiment, the invention provides administration of a PDE1 inhibitor for use in the methods of treatment and prophylaxis described herein, wherein the inhibitor is a compound according to the following:

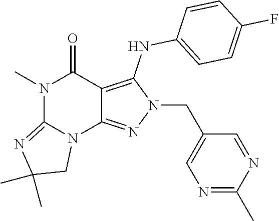 [SMILES: Cc1ncc(Cn2nc3c(c2Nc2ccc(F)cc2)C(=O)N(C)C2=NC(C)(C)CN23)cn1]

in free or pharmaceutically acceptable salt form.

In still another embodiment, the invention provides administration of a PDE1 inhibitor for use in the methods of treatment and prophylaxis described herein, wherein the inhibitor is a compound according to the following:

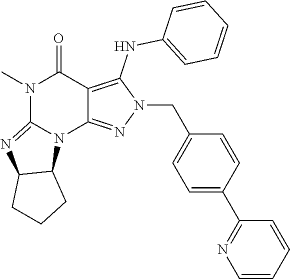 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.

In one embodiment, selective PDE1 inhibitors of the any of the preceding formulae (e.g., Formula I, Ia, II, III and/or IV) are compounds that inhibit phosphodiesterase-mediated (e.g., PDE1-mediated, especially PDE1B-mediated) hydrolysis of cGMP and/or cAMP, e.g., the preferred compounds have an IC₅₀ of less than 1 M, preferably less than 500 nM, preferably less than 50 nM, and preferably less than 5 nM in an immobilized-metal affinity particle reagent PDE enzyme assay, in free or salt form.

In other embodiments, the invention provides administration of a PDE1 inhibitor for use in the methods of treatment and prophylaxis described herein, wherein the PDE1 inhibitor is a compound according to the following:

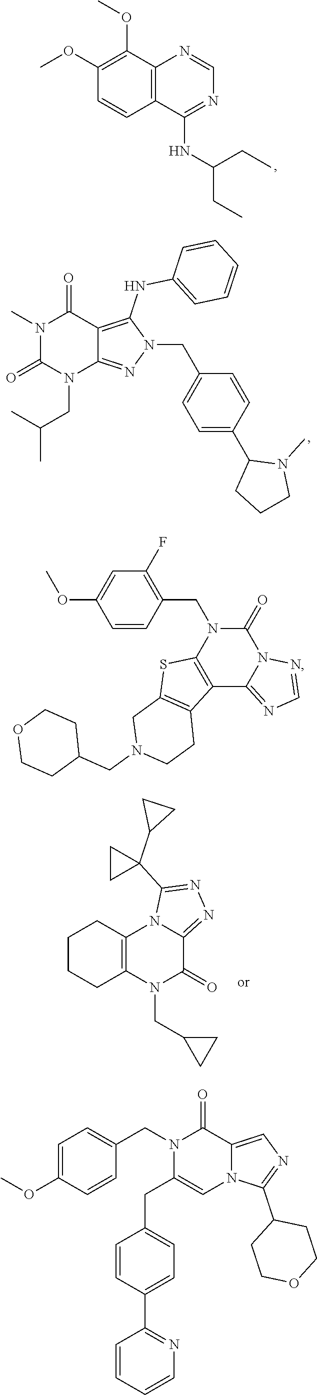

Further examples of PDE1 inhibitors suitable for use in the methods and treatments discussed herein can be found in International Publication WO2006133261A2; U.S. Pat. Nos. 8,273,750; 9,000,001; 9,624,230; International Publication WO2009075784A1; U.S. Pat. Nos. 8,273,751; 8,829,008; 9,403,836; International Publication WO2014151409A1, U.S. Pat. Nos. 9,073,936; 9,598,426; 9,556,186; U.S. Publication 2017/0231994A1, International Publication WO2016022893A1, and U.S. Publication 2017/0226117A1, each of which are incorporated by reference in their entirety.

Still further examples of PDE1 inhibitors suitable for use in the methods and treatments discussed herein can be found in International Publication WO2018007249A1; U.S. Publication 2018/0000786; International Publication WO2015118097A1; U.S. Pat. No. 9,718,832; International Publication WO2015091805A1; U.S. Pat. No. 9,701,665; U.S. Publication 2015/0175584A1; U.S. Publication 2017/0267664A1; International Publication WO2016055618A1; U.S. Publication 2017/0298072A1; International Publication WO2016170064A1; U.S. Publication 2016/0311831A1; International Publication WO2015150254A1; U.S. Publication 2017/0022186A1; International Publication WO2016174188A1; U.S. Publication 2016/0318939A1; U.S. Publication 2017/0291903A1; International Publication WO2018073251A1; International Publication WO2017178350A1; and U.S. Publication 2017/0291901A1; each of which are incorporated by reference in their entirety. In any situation in which the statements of any documents incorporated by reference contradict or are incompatible with any statements made in the present disclosure, the statements of the present disclosure shall be understood as controlling.

If not otherwise specified or clear from context, the following terms herein have the following meanings:
(a) “Alkyl” as used herein is a saturated or unsaturated hydrocarbon moiety, preferably saturated, preferably having one to six carbon atoms, which may be linear or branched, and may be optionally mono-, di- or tri-substituted, e.g., with halogen (e.g., chloro or fluoro), hydroxy, or carboxy.
(b) “Cycloalkyl” as used herein is a saturated or unsaturated nonaromatic hydrocarbon moiety, preferably saturated, preferably comprising three to nine carbon atoms, at least some of which form a nonaromatic mono- or bicyclic, or bridged cyclic structure, and which may be optionally substituted, e.g., with halogen (e.g., chloro or fluoro), hydroxy, or carboxy. Wherein the cycloalkyl optionally contains one or more atoms selected from N and O and/or S, said cycloalkyl may also be a heterocycloalkyl.
(c) “Heterocycloalkyl” is, unless otherwise indicated, saturated or unsaturated nonaromatic hydrocarbon moiety, preferably saturated, preferably comprising three to nine carbon atoms, at least some of which form a nonaromatic mono- or bicyclic, or bridged cyclic structure, wherein at least one carbon atom is replaced with N, O or S, which heterocycloalkyl may be optionally substituted, e.g., with halogen (e.g., chloro or fluoro), hydroxy, or carboxy.
(d) “Aryl” as used herein is a mono or bicyclic aromatic hydrocarbon, preferably phenyl, optionally substituted, e.g., with alkyl (e.g., methyl), halogen (e.g., chloro or fluoro), haloalkyl (e.g., trifluoromethyl), hydroxy, carboxy, or an additional aryl or heteroaryl (e.g., biphenyl or pyridylphenyl).
(e) “Heteroaryl” as used herein is an aromatic moiety wherein one or more of the atoms making up the aromatic ring is sulfur or nitrogen rather than carbon, e.g., pyridyl or thiadiazolyl, which may be optionally substituted, e.g., with alkyl, halogen, haloalkyl, hydroxy or carboxy.
(f) For ease of reference, the atoms on the pyrazolo-pyrimidine core of the Compounds of the Disclosure are numbered in accordance with the numbering depicted in Formula I, unless otherwise noted.
(g) It is intended that wherein the substituents end in “ene,” for example, alkylene, phenylene or arylalkylene, said substitutents are intended to bridge or be connected to two other substituents. Therefore, methylene is intended to be —CH₂— and phenylene intended to be —C₆H₄— and arylalkylene is intended to be —C₆H₄—CH₂— or —CH₂—C₆H₄—.

Compounds of the Disclosure, e.g., substituted 4,5,7,8-tetrahydro-2H-imidazo[1,2-a]pyrrolo[3,4-e]pyrimidine or 4,5,7,8,9-pentahydro-2H-pyrimido[1,2-a]pyrrolo[3,4-e]pyrimidine, e.g., Compounds of Formula I, Ia, II, III or IV, may exist in free or salt form, e.g., as acid addition salts. In this specification unless otherwise indicated, language such as “Compounds of the Disclosure” is to be understood as embracing the compounds in any form, for example free or acid addition salt form, or where the compounds contain acidic substituents, in base addition salt form. The Compounds of the Disclosure are intended for use as pharmaceuticals, therefore pharmaceutically acceptable salts are preferred. Salts which are unsuitable for pharmaceutical uses may be useful, for example, for the isolation or purification of free Compounds of the Disclosure or their pharmaceutically acceptable salts, are therefore also included.

Compounds of the Disclosure, encompassing any of the compounds disclosed herein, e.g., optionally substituted 4,5,7,8-tetrahydro-(optionally 4-thioxo or 4-imino)-(1H or 2H)-imidazo[1,2-a]pyrazolo[4,3-e]pyrimidine or 4,5,7,8,9-pentahydro-(1H or 2H)-pyrimido[1,2-a]pyrazolo[4,3-e]pyrimidine compounds, e.g., (1 or 2 and/or 3 and/or 5)-substituted 4,5,7,8-tetrahydro-1H-imidazo[1,2-a]pyrazolo[4,3-e]pyrimidine, 4,5,7,8-tetrahydro-2H-imidazo[1,2-a]pyrazolo[4,3-e]pyrimidine, 4,5,7,8-tetrahydro-(1H or 2H)-pyrimido[1,2-a]pyrazolo[4,3-e]pyrimidine-4(5H)-imine, 7,8-dihydro-1H-imidazo[1,2-a]pyrazolo[4,3-e]pyrimidine-4(5H)-thione or 7,8-dihydro-2H-imidazo[1,2-a]pyrazolo[4,3-e]pyrimidine-4(5H)-thione compounds, e.g., Compounds of Formula III, or Compound of Formula IV as described herein, may exist in free or salt form, e.g., as acid or base addition salts.

Compounds of the Disclosure may in some cases also exist in prodrug form. A prodrug form is compound which converts in the body to a Compound of the Disclosure. For example, when the Compounds of the Disclosure contain hydroxy or carboxy substituents, these substituents may form physiologically hydrolysable and acceptable esters. As used herein, “physiologically hydrolysable and acceptable ester” means esters of Compounds of the Disclosure which are hydrolysable under physiological conditions to yield acids (in the case of Compounds of the Disclosure which have hydroxy substituents) or alcohols (in the case of Compounds of the Disclosure which have carboxy substituents) which are themselves physiologically tolerable at doses to be administered. Therefore, wherein the Compound of the Disclosure contains a hydroxy group, for example, Compound-OH, the acyl ester prodrug of such compound, i.e., Compound-O—C(O)—C₁₋₄alkyl, can hydrolyze in the body to form physiologically hydrolysable alcohol (Compound-OH) on the one hand and acid on the other (e.g., HOC(O)—C₁₋₄alkyl). Alternatively, wherein the Compound of the Disclosure contains a carboxylic acid, for example, Compound-C(O)OH, the acid ester prodrug of such compound, Compound-C(O)O—C₁₋₄alkyl can hydrolyze to form Compound-C(O)OH and HO—C₁₋₄alkyl. As will be appreciated the term thus embraces conventional pharmaceutical prodrug forms.

In another embodiment, the disclosure further provides a pharmaceutical composition comprising a Compound of the Disclosure, in free or pharmaceutically acceptable salt form, in admixture with a pharmaceutically acceptable carrier.

In another embodiment, the disclosure further provides a pharmaceutical composition comprising a Compound of the Disclosure, in free, pharmaceutically acceptable salt or prodrug form, in admixture with a pharmaceutically acceptable carrier.

In some embodiments, the Compounds of the Disclosure may be modified to affect their rate of metabolism, e.g., to increase half life in vivo. In some embodiments, the compounds may be deuterated or fluorinated to reduce the rate of metabolism of the compounds disclosed herein.

In some embodiments, the compounds are provided in the form of a long acting depot composition for administration by injection to provide sustained release. In some embodiments, the solid drug for oral administration or as a depot may be in a suitable polymer matrix to provide delayed release of the active compound.

The Compounds of the Disclosure and their pharmaceutically acceptable salts may be made using the methods as described and exemplified herein and by methods similar thereto and by methods known in the chemical art. If not commercially available, starting materials for these processes may be made by procedures, which are selected from the chemical art using techniques which are similar or analogous to the synthesis of known compounds. Starting materials and methods of making Compounds of the Disclosure are described in the patent applications cited and incorporated by reference above.

The Compounds of the Disclosure include their enantiomers, diastereoisomers and racemates, as well as their polymorphs, hydrates, solvates and complexes. Some individual compounds within the scope of this disclosure may contain double bonds. Representations of double bonds in this disclosure are meant to include both the E and the Z isomer of the double bond. In addition, some compounds within the scope of this disclosure may contain one or more asymmetric centers. This disclosure includes the use of any of the optically pure stereoisomers as well as any combination of stereoisomers.

It is also intended that the Compounds of the Disclosure encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the Compounds of the Disclosure may be replaced with deuterium (a stable isotope which is non-raradioactive). Examples of known stable isotopes include, but not limited to, deuterium, ¹³C, ¹⁵N, ¹⁸O. Alternatively, unstable isotopes, which are radioactive isotopes which contain additional neutrons compared to the abundant nuclides of the same species (i.e., element), e.g., ¹²³I, ¹³¹I, ¹²⁵I, ¹¹C, ¹⁸F, may replace the corresponding abundant species of I, C and F. Another example of useful isotope of the compound of the disclosure is the ¹¹C isotope. These radio isotopes are useful for radio-imaging and/or pharmacokinetic studies of the compounds of the disclosure.

### Drug-Eluting Devices

Improved drug delivery devices are disclosed for administering a drug or other active agent to the ocular region in order to treat an ophthalmic disease, disorder or injury. In one aspect, the device is a drug eluting contact lens which can be applied to a patient's cornea. In some embodiments, the contact lens device comprises a drug release matrix comprising at least one drug (e.g., a PDE1 inhibitor) and optionally a hydrogel lens material encapsulating the drug release material to provide controlled release of the drug over an extended period of time. In some embodiments, the structure beneficially enables the contact lens to be loaded with a high level of drug, while simultaneously providing for prolonged and controlled release. The contact lens device advantageously can provide sustained release of therapeutically or prophylactically effective amounts of the drug over a sustained period.

Thus, in some embodiments, the present disclosure provides for a drug eluting contact lens [Device 1] comprising a pharmaceutically effective amount of a PDE1 inhibitor encapsulated in a drug release matrix, wherein the drug eluting contact lens is configured to release the PDE1 inhibitor over a sustained period of time. For example, the present disclosure provides for the following embodiments of Device 1:
1.1 Device 1, wherein the device is implanted for the treatment or prophylaxis of an ophthalmic disease, disorder or injury.
1.2 Device 1 or 1.1, wherein the PDE1 inhibitor is a compound according to Formula I, Ia, II, III or IV.
1.3 Any preceding Device, wherein the PDE1 inhibitor comprises (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one:

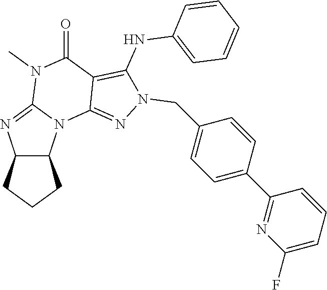 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
1.4 Any foregoing Device, wherein the PDE1 inhibitor comprises 7,8-dihydro-2-(4-acetylbenzyl)-3-(4-fluorophenylamino)-5,7,7-trimethyl-[2H]-imidazo-[1,2-a]pyrazolo[4,3-e]pyrimidin-4(5H)-one:

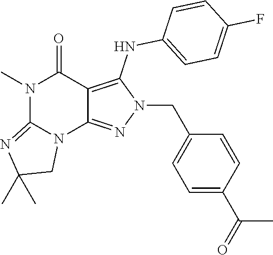 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.
1.5 Any foregoing Device, wherein the PDE1 inhibitor comprises 3-((4-fluorophenyl)amino)-5,7,7-trimethyl-2-((2-methylpyrimidin-5-yl)methyl)-7,8-dihydro-2H-imidazo[1,2-a]pyrazolo[4,3-e]pyrimidin-4(5H)-one

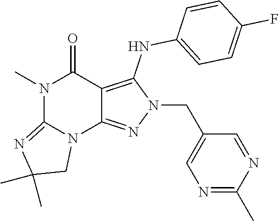 [SMILES: Cc1ncc(Cn2nc3c(c2Nc2ccc(F)cc2)C(=O)N(C)C2=NC(C)(C)CN23)cn1]

in free or pharmaceutically acceptable salt form.
1.6 Any foregoing Device, wherein the PDE1 inhibitor comprises (6aR,9aS)-5,6a,7,8,9,9a-hexahydro-5-methyl-3-(phenylamino)-2-((4-(pyridin-2-yl)phenyl)methyl)-cyclopent[4,5]imidazo[1,2-a]pyrazolo[4,3-e]pyrimidin-4(2H)-one:

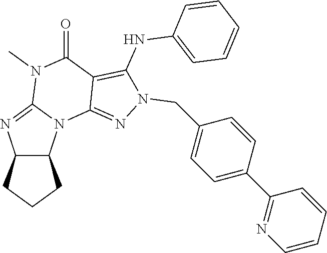 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.
1.7 Any foregoing Device, wherein the drug release matrix comprises a biodegradable polymer, a nondegradable polymer or a hydrogel or a combination thereof.
1.8 Any foregoing Device, wherein the drug release matrix comprises or consists of a biodegradable polymer.
1.9 Any foregoing Device, wherein the drug release matrix comprises a degradable polymer selected from the group consisting of poly(lactic-co-glycolic) acid (“PLGA”), polylactide, polyglycolide, polycaprolactone, or other polyesters, poly(orthoesters), poly(aminoesters), polyanhydrides, polyorganophosphazenes, or combinations thereof.
1.10 Any foregoing Device, wherein the drug release matrix comprises or consists of a non-degradable polymer.
1.11 Any foregoing Device, wherein the drug release matrix comprises non-degradable polymer selected from the group consisting of ethyl cellulose, poly(butyl acrylate), poly(urethanes), silicone resins, nylon, ammonium polyacrylate, acrylamide copolymers, acrylate/acrylamide copolymers, acrylate/ammonium acrylate copolymers, acrylate/alkyl acrylate copolymers, acrylate/carbamate copolymers, acrylate/dimethylaminoethyl methacrylate copolymers, ammonium acrylate copolymers, styrene/acrylate copolymers, vinyl acetate/acrylate copolymers, aminomethylpropanol/acrylate/dimethylaminoethylmethacrylate copolymers, or combinations thereof.
1.12 Any foregoing Device, wherein the drug release matrix comprises or consists of a hydrogel.
1.13 Any foregoing Device, wherein the drug release matrix comprises a hydrogel selected from polyhydroxyethylmethacrylate (pHEMA), a silicone, agarose, alginate, chitosan, and hyaluronic acid.
1.14 Any preceding Device, wherein the drug release matrix comprises a biodegradable polymer, a nondegradable polymer or a hydrogel or a combination thereof in an amount of about 1-99% by weight of the total weight of the drug release matrix, about 50-90% by weight of the total weight of the drug release matrix, or about 60-80% by weight of the total weight of the drug release matrix.
1.15 Any foregoing Device, wherein the drug release matrix is in the form of a film configured to fit over the cornea of a subject in need thereof.
1.16 The preceding Device, wherein the drug release matrix is loaded with a concentration of the drug toward perimeter of the device, such that the drug release matrix substantially does not release the drug from a portion of the device aligning over a subject's pupil.
1.17 Any foregoing Device, wherein the drug-eluting contact lens further comprises a lens matrix that a) forms a layer abutting the drug release matrix or b) at least partially encapsulates the drug release matrix.
1.18 Any foregoing Device, wherein the drug eluting contact lens comprises a first layer comprising the drug release matrix and a second layer comprising a non-eluting material (e.g., a lens matrix), such that when in use the first layer faces toward the surface of the eye.
1.19 The preceding Device, wherein the non-eluting material is a non-degradable polymer or a hydrogel.
1.20 The two preceding Devices, wherein the non-eluting material forms a lens matrix.
1.21 Any preceding Device, wherein the drug-eluting contact lens comprises at least one additional active agent.
1.22 The preceding Device wherein, the additional active agent is an intraocular pressure-lowering agent, a growth factor; an angiogenic agent; an anti-inflammatory agent; an anti-infective agent such as antibacterial agents, an antiviral agent, an antifungal agent, and agents that inhibit protozoan infections; an antineoplastic agent; an anesthetic; an anti-cancer composition; an autonomic agent; a steroid (e.g., corticosteroid); a non-steroidal anti-inflammatory drug (NSAIDs); an antihistamine; a mast-cell stabilizer; an immunosuppressive agent; an antimitotic agent; anti-oxidant; nitroxide generating agenty or combinations thereof.
1.23 The two preceding Devices, wherein the additional active agent is an intraocular pressure-lowering agent.
1.24 The preceding Device, wherein the intraocular pressure-lowering agent is an adrenergic agonist, a beta-adrenergic antagonist, a prostaglandin or prostaglandin analog or a muscarinic analog, or an agent that raises cyclic nucleotides, a sympathomimetic, a miotic agent, a carbonic anhydrase inhibitor, a prostanoid, physostigmine, bimatoprost, brimonidine tartrate, or brimonidine tartrate/timolol maleate, or a combination thereof.
1.25 The preceding Device, wherein the prostaglandin is travoprost, latanoprost, bimatroprost, unoprostone, and unoprostone isopropyl.
1.26 The preceding Device, wherein the alpha adrenergic agonist is brimonidine, apraclonidine or dipivefrin.
1.27 The preceding Device, wherein the muscarinic agonist is pilocarpine.
1.28 Any preceding Device, wherein the device contains 0.1 μg to 100 mg of the PDE1 inhibitor, 1 μg to 100 mg of the PDE1 inhibitor, 10 μg to 100 mg of the PDE1 inhibitor, 50 μg to 100 mg of the PDE1 inhibitor, 100 μg to 100 mg of the PDE1 inhibitor, 500 μg to 50 mg of the PDE1 inhibitor, 1 mg to 50 mg of the PDE1 inhibitor, 5 mg to 50 mg of the PDE1 inhibitor, 10 mg to 40 mg of the PDE1 inhibitor, 20 mg to 35 mg of the PDE1 inhibitor, e.g., 10 mg to 30 mg of the PDE1 inhibitor.
1.29 Any preceding Device, wherein the PDE1 inhibitor is released at a constant rate (e.g., a zero order release profile) or at a higher initial concentration that tapers over time (e.g., a first order release profile) over a sustained period of time.
1.30 Any preceding Device, wherein the PDE1 inhibitor is released at a constant rate (e.g., a zero order release profile) over a sustained period of time.
1.31 Any preceding Device, wherein the PDE1 inhibitor is released at a rate of rate of 0.001 μg per hour, 0.005 μg per hour, 0.01 μg per hour, 0.05 μg per hour, 0.1 μg per hour, 0.5 μg per hour, 1 μg per hour, 5 μg per hour, 10 μg per hour, 20 μg per hour, 50 μg per hour, 100 μg per hour, 500 μg per hour, 1 mg per hour, or 5 mg per hour.
1.32 Any preceding Device, wherein the contact lens provides sustained release of the PDE1 inhibitor for at least 1 hour, at least 4 hours, at least 8 hours, at least 16 hours, at least 24 hours, at least 72 hours, at least 168 hours, at least 336 hours, at least 720 hours, at least 1440 hours, at least 2160 hours, at least 2400 hours, at least 2880 hours, at least 3600 hours, at least 4800 hours, at least 7200 hours, at least 9600 hours, or even longer.
1.33 Any preceding device, wherein the contact lens provides sustained release of the PDE1 inhibitor for 24 hours, 2 weeks, one month or three months.
1.34 Any preceding Device, further comprising at least one plasticizer.
1.35 The preceding Device, wherein the plasticizer is a pluronic/poloxamer non-ionic surfactant or a lipid.
1.36 The preceding Device, wherein the plasticizer is Span 20, Tween 20, propylene glycol, sorbitan monolaurate and propylene glycol, and combinations thereof.
1.37 Any preceding Device, wherein the PDE1 inhibitor is present in an amount of about 1% to about 99% by weight of the drug release matrix, from about 5% to about 70% by weight, or from about 10% to about 30% by weight.
1.38 Any preceding Device, wherein the disease, disorder or injury is optic nerve injury or trauma, retinal injury or trauma, blindness consequent to diabetes, optic neuritis (e.g., optic neuritis consequent to infection or optic neuritis consequent to an autoimmune disorder such as multiple sclerosis or systemic lupus erythematosus), glaucoma, infections including viral or bacterial, cancers, or elevated intraocular pressure.
1.39 The preceding Device, wherein the disease, disorder or injury is optic nerve injury or trauma.
1.40 Device 1.38, wherein the disease, disorder or injury is retinal injury or trauma.
1.41 Device 1. 38, wherein the disease, disorder or injury is blindness consequent to diabetes.
1.42 Device 1. 38, wherein the disease, disorder or injury is optic neuritis.
1.43 The preceding Device, wherein optic neuritis is consequent to infection or an autoimmune disorder such as multiple sclerosis or systemic lupus erythematosus.
1.44 Device 1. 38, wherein the disease, disorder or injury is glaucoma or elevated intraocular pressure.
1.45 Device 1. 38, wherein the disease is glaucoma.
1.46 Any preceding Device, wherein the contact lens is removable.
1.47 Any preceding Device, wherein the contact lens is disposable.
1.48 Any preceding Device, wherein the contact lens is left on the subject's eye until the entire drug load of PDE1 inhibitor has been released.
1.49 Any preceding Device, wherein the contact lens is left on the subject's eye until the contact lens fully degrades.

Improved drug delivery devices are disclosed for administering a drug or other active agent to the ocular region. In one aspect, the device is a drug eluting contact lens which can be applied to a patient's cornea. In some embodiments, the contact lens device comprises a drug release matrix comprising at least one drug (e.g., a PDE1 inhibitor) and a lens matrix to provide controlled release of the drug over a sustained period of time. In some embodiments, the structure beneficially enables the contact lens to be loaded with a high level of drug, while simultaneously providing for prolonged and controlled release. The contact lens device advantageously can provide sustained release of therapeutically or prophylactically effective amounts of the drug over an extended period.

In some embodiments, the drug delivery device includes a drug release matrix comprising at least one active agent (e.g, a PDE1 inhibitor) and a lens matrix either forming a layer that abuts the drug release matrix or encapsulates the drug release matrix. The device generally may be provided in any configuration or geometry that would be suitable for a given ocular or non-ocular use.

In certain embodiments, the contact lens device may be provided with structural features to impart directional control over the release of the drug. For example, the contact lens device may include micro-perforations, selected areas of varying thickness and permeability, or any combination thereof, which may for example facilitate mass transport of the drug by reducing diffusional distances or enabling bulk fluid flow through portions of the device rather than having to rely solely on diffusional mass transport.

In some embodiments, the contact lens device may deliver more than one type of drug simultaneously. In one embodiment, the contact lens device includes a second drug, which may be incorporated into the drug release matrix, the lens matrix, or both. It should be understood that the contact lens may contain a third drug, fourth drug, or even more drugs.

In some embodiments, the drug release matrix may be configured as a film. As used herein, the term “film” means a thin, preferably flexible, monolithic sheet of material having a thickness from 50 nm to 500 μm. In one embodiment, the film is from about 1 μm to 500 μm. In one embodiment, the film has a thickness from about 10 μm to about 100 μm. A layer comprising a collection of microparticles is not a film; however, the drug release matrix may comprise a collection of particles such as microparticles and/or nanoparticles.

Generally, the drug release matrix of the device described herein may be fashioned into various shapes and sizes. In certain embodiments, the drug release matrix is circular or ring shaped, or semi-circular, crescent-shaped, or arch-shaped. In one embodiment, the drug release matrix includes an aperture about the optical axis of the contact lens. In one embodiment, the drug release matrix may have a multi-layer structure. In some cases, the drug release matrix may include a coating on one or both sides, for example, with a polymer containing no drug or less or more drug than the drug release matrix. The drug release matrix may comprise a single film or a stack of two or more films. In various embodiments, the drug release matrix is loaded with a concentration of the drug toward the perimeter of the device, such that the drug release matrix substantially does not release the drug from a portion of the device aligning over a subject's pupil.

The device may be used to deliver essentially any active agent (e.g., a drug), including for example small molecule drugs, proteins, nucleic acids, polysaccharides, and biologics. The drug may be any agent capable of providing a therapeutic benefit. In an embodiment, the drug is a known drug, or drug combination, effective for treating diseases and disorders of the eye. In non-limiting, exemplary embodiments, the drug is an antiinfective agent (e.g., an antibiotic or antifungal agent), an anesthetic agent, an anti-VEGF agent, an anti-inflammatory agent, a biological agent (such as RNA), an intraocular pressure reducing agent (i.e., a glaucoma drug), or a combination thereof. Non-limiting examples of drugs are provided below.

The polymer in the drug release matrix is essentially any biocompatible polymer, co-polymer, terpolymer, or polymer blend. In one aspect, the polymer of the drug release matrix is biodegradable. In one embodiment, the products of the polymers' degradation should not pose a health risk to the ocular region. Biodegradability of the polymer in the drug release matrix is typically not a primary consideration except to the extent that the degradation may promote the desired release of the drug. Generally, both biodegradable and non-biodegradable polymers may be used to create drug polymer films that function desirably in the devices described herein.

In one embodiment, the polymer of the drug release matrix is biodegradable. For example, the polymer of the drug release matrix may be poly(lactic-co-glycolic) acid (“PLGA”), polylactide, polyglycolide, polycaprolactone, or other polyesters, poly(orthoesters), poly(aminoesters), polyanhydrides, polyorganophosphazenes, or any combination thereof. Other biodegradable polymers known to those skilled in the art may also be applied and selected based on the desired mechanical properties and polymer-drug interaction.

Further examples of useful biodegradable polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate. Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyvinyl alcohol, polyesters, polyethers and combinations thereof which are biocompatible and may be biodegradable and/or bioerodible. Some preferred characteristics of the polymers or polymeric materials for use in the present invention may include biocompatibility, compatibility with the selected therapeutic agent, ease of use of the polymer in making the drug delivery systems of the present invention, a half-life in the physiological environment of at least about 6 hours, preferably greater than about one day, and water insolubility.

The biodegradable polymeric materials which are included to form the matrix are desirably subject to enzymatic or hydrolytic instability. Water soluble polymers or poorly water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Different molecular weights of the same or different polymeric compositions may be included in the drug release matrix to modulate the release profile. For example, the relative average molecular weight of the polymer will preferably range from about 4 to about 25 kD, more preferably from about 5 to about 20 kD, and most preferably from about 5 to about 15 kD.

In some embodiments, copolymers of glycolic acid and lactic acid are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the drug release matrix. The percentage of polylactic acid in the polylactic acid polyglycolic acid (PLGA) copolymer can be 0-100%, preferably about 15-85%, more preferably about 35-65%. In some implants, a 50/50 PLGA copolymer is used.

In another embodiment, the polymer of the drug release matrix is non-degradable. For example, the polymer of the drug release matrix may be ethyl cellulose, poly(butyl acrylate), poly(urethanes), silicone resins, nylon, ammonium polyacrylate, acrylamide copolymers, acrylate/acrylamide copolymers, acrylate/ammonium acrylate copolymers, acrylate/alkyl acrylate copolymers, acrylate/carbamate copolymers, acrylate/dimethylaminoethyl methacrylate copolymers, ammonium acrylate copolymers, styrene/acrylate copolymers, vinyl acetate/acrylate copolymers, aminomethylpropanol/acrylate/dimethylaminoethylmethacrylate copolymers, or any combination thereof. Other non-degradable polymers known to those skilled in the art may also be applied and selected based on the desired mechanical properties and polymer-drug interaction.

In some embodiments, the drug release matrix may comprise a hydrogel. Examples of hydrogels include, but are not limited to, polyhydroxyethylmethacrylate (pHEMA), a silicone, agarose, alginate, chitosan, and hyaluronic acid. Other hydrogels known to those skilled in the art may also be applied and selected based on the desired mechanical properties and hydrogel-drug interaction. The drug release matrix may, in some cases, form a gel within a pH range. In another embodiments, the drug release matrix may transition between a liquid and a gel at a critical temperature.

Generally, the lens matrix, when present, may be any biocompatible material suitable for ocular or non-ocular medical uses. In one embodiment, the lens matrix is a hydrogel known in the art of soft contact lenses. For example, in various embodiments, the lens matrix may comprise polyhydroxyethylmethacrylate (pHEMA), a silicone, or a composite comprising silicone dispersed in a hydrogel. In one embodiment, the hydrogel comprises polyhydroxyethylmethacrylate (pHEMA) or co-polymers thereof. In another embodiment, the hydrogel comprises a silicone hydrogel. In still another embodiment, the hydrogel comprises hyaluronic acid. The hydrogels may be cross-linked using methods and/or materials known in the art, which are suitable for use with the ocular tissues. In one embodiment, the cross-linking agent is ethyleneglycol dimethacrylate (EGDMA). In an alternative embodiment, the lens matrix comprises a non-hydrogel material. The non-hydrogel material may have suitable oxygen, water, and drug permeability properties to permit its use as a contact lens.

In various embodiments, the drug release matrix controls the release kinetics of the drug from the drug release matrix, although the drug release matrix may provide the rate-limiting control in certain embodiments, particularly for sustained release. Both the encapsulating lens matrix and the polymer of the drug release matrix may be adjusted, for example as described herein, to obtain the desired release of the at least one drug from the device. In one embodiment, the controlled release of the at least one drug from the contact lens is substantially zero-order or first-order. In certain embodiments, the contact lens provides sustained release of the at least one drug for at least 1 hour, at least 4 hours, at least 8 hours, at least 16 hours, at least 24 hours, at least 72 hours, at least 168 hours, at least 336 hours, at least 720 hours, at least 1440 hours, at least 2160 hours, at least 2400 hours, at least 2880 hours, at least 3600 hours, at least 4800 hours, at least 7200 hours, at least 9600 hours, or even longer. In other embodiments, the contact lens provides release of the at least one drug at therapeutically effective amounts for at least 168 hours, at least 336 hours, at least 720 hours, at least 1440 hours, at least 2160 hours, at least 2400 hours, at least 2880 hours, at least 3600 hours, at least 4800 hours, at least 7200 hours, at least 9600 hours, or even longer.

As used herein, the period of time in which a device releases a drug refers to the period of time in which the device is releasing a drug in a subject or in an environment that mimics the environment in a subject. As a non-limiting example, release of a drug by a device for a 24-hour period of time may be achieved by a subject wearing a contact lens continuously for 24 hours or intermittently for a total period of 24 hours (e.g., by wearing a contact lens for 1 hour per day for 24 days).

Release of drug from the contact lens device may be controlled, in part, by the composition of the polymer in the drug release matrix. For example, increasing or decreasing the rate of release of the drug may be accomplished by altering the polymer. If the polymer is a co-polymer, such alteration may include changing the ratio of the monomers in the copolymer. In an exemplary embodiment, the polymer in the drug release matrix is PLGA. Increasing the ratio of lactide to glycolide generally will slow the release of the drug from the drug release matrix. To illustrate, polylactic acid, which contains no glycolide, may provide the slowest release system of this embodiment, whereas polyglycolic acid, which contains no lactide, may provide the fastest release system of this embodiment.

In addition, or in the alternative, release of drug from the contact lens device may be controlled, in part, by the selection of the ratio of polymer to drug in the drug release matrix. While maintaining a constant mass of polymer, the amount of the drug in the drug release matrix may be reduced so that drug release matrix with polymer to drug ratios of 1:2, 1:4, 1:8, 1:16; 1:32; 1:64; 1:128; 1:256; 1:512; or any other desirable ratio may be obtained. If a higher ratio of polymer is needed to attain the desired release of the drug, the potency of the drug may be adjusted. Generally, increasing the potency of the drug decreases the mass of the drug payload that must be incorporated into the drug release matrix. Furthermore, increasing the potency of the drug may reduce the footprint of the drug release matrix within the device, thereby enhancing flexibility, oxygen permeability, or cosmesis.

In some embodiments, the contact lens may contain at least 0.1 micrograms of drug, at least 1 microgram of a drug, at least 10 micrograms of a drug, at least 50 micrograms of a drug, at least 100 micrograms of a drug, at least 500 micrograms of a drug, at least 1 mg of a drug, at least 5 mg of a drug, at least 10 mg of a drug, at least 20 mg of a drug, at least 50 mg of a drug, at least 100 mg of a drug, or even more.

The rate of drug release from the device may be altered by changing the polymer concentration in the film. In one aspect, this concentration may be adjusted by altering the solvent casting procedure as described herein. The rate of drug release from the device also may be altered by casting multiple polymer-drug layers of different compositions. In one such embodiment, a lower layer may be cast with a low drug:polymer ratio, a middle layer cast with a high drug:polymer ratio, and an upper layer cast with a low drug:polymer ratio. In such an embodiment, the film would contain a high total amount of drug while maintaining a low surface permeability for drug release to prolong the release period. In another embodiment, different polymers with different solubilities may be used such that the casting of each subsequent layer has no significant effect on the preceding layer(s). In one specific embodiment, different polymers may be used to prepare the different layers. In another embodiment, the relative thicknesses of the layers can be altered to change the rate of drug release.

In some embodiments, the hydrogel may be altered to influence the release of the drug from the device. In some embodiments, the hydrogel at least partly modulates the release of drugs from the device. In one aspect, the hydrogel may be made more hydrophobic in order to alter the release of the drug from the device. The hydrogel material may be co-polymerized with a small percentage of hydrophobic acrylates such as 1,3-butylene glycol diacrylate, isooctyl acrylate, and lauryl acrylate. Alternately, monomers with specific or non-specific affinities to the drug may be co-polymerized into the hydrogel to slow the release of the drug from the device. The release of drugs with cationic functional groups, such as timolol, may be slowed by the presence of acid groups in the hydrogel. In one embodiment, an acidic monomer such as (but not limited to) acrylic acid, methacrylic acid, fumaric acid, vinylacetic acid, itaconic acid, or maleic acid may be co-polymerized into the hydrogel.

In some embodiments, the rate of drug release from the contact lens is at least 0.001 micrograms per hour, at least 0.005 micrograms per hour, at least 0.01 micrograms per hour, at least 0.05 micrograms per hour, at least 0.1 micrograms per hour, at least 0.5 micrograms per hour, at least 1 microgram per hour, at least 5 micrograms per hour, at least 10 micrograms per hour, at least 20 micrograms per hour, at least 50 micrograms per hour, at least 100 micrograms per hour, at least 500 micrograms per hour, at least 1 mg per hour, at least 5 mg per hour, or even more. It should be understood that a contact lens may release a drug at any of these rates even if releasing the drug for less than 1 hour. As a non-limiting example, a contact lens may be worn by a subject for less than 1 hour yet still release a drug at a rate measured in terms of units of drug released per hour.

The device may be manufactured in a manner that promotes the release of the drug toward or away from the cornea or sclera. In one embodiment, the device may be produced so that that the portion of the encapsulating hydrogel lens matrix on one side of the drug release matrix is thinner relative to the portion of the lens matrix on the other side of the drug release matrix. In another embodiment, a certain portion of the hydrogel lens matrix may be made more hydrophobic or hydrophilic to promote the unidirectional release of the drug from the device. Alternatively, a certain portion of the encapsulating hydrogel lens matrix may be made more gas soluble or gas permeable in order to promote the unidirectional release of the drug from the device. In yet another embodiment, portions of the encapsulating hydrogel lens matrix may be perforated in order to promote the unidirectional release of the drug from the device. Typically, the perforations are micro-perforations.

In another aspect, the controlled release device described herein is implanted into the patient's cornea. In yet another embodiment, the device described herein may be sutured to the sclera of the patient's eye. In still another embodiment, the device may comprise an intraocular lens. Typically, but not always, this embodiment of the device comprises an optic portion and a haptics portion. An intraocular lens may be an optic lens, a haptic lens, or both an optic and haptic lens. In yet another embodiment, the device described herein may comprise a glaucoma tube. For example, the device may be a glaucoma drainage device.

In still another aspect, the drug delivery device described herein may be adapted for use in areas of the human or animal body besides the eye. Generally, the device may be used in any portion of the body that would benefit from the controlled release of a drug. The device may be inserted, sutured, or placed onto various tissue structures or into various vesicles or cavities where local controlled drug release is desired.

Aside from a PDE1 inhibitor according to the present disclosure, the device may include one or more anti-infective agents for controlled release. Non-limiting examples of suitable anti-infective agents include ciprofloxacin, moxifloxacin, galtfloxacin, vancomyacin, tobramyacin, or a combination thereof. Other known anti-infective agents may be incorporated into and released from the contact lens device. In still other embodiments, the contact lens may include a second drug, such as a steroid (e.g., corticosteroids), non-steroidal anti-inflammatory drugs (NSAIDs), local anesthetic agent, or other drug, for co-administration with the anti-infective agent. In one embodiment, a contact lens drug delivery device is provided which includes a combination of an anti-infective and a steroid. The local anesthetic agent may be an aminoamide, an aminoester, or a mixture thereof. Combinations of different aminoamides or combinations of different aminoesters are envisioned. Representative examples of possible aminoamides include lidocaine, prilocalne, mepivacaine, and ropivacaine. Representative examples of possible aminoesters include benzocaine, procaine, proparacaine, and tetracaine.

In some embodiments, plasticizers may be incorporated into the device to alter the drug release characteristics of the device. As used herein, the plasticizer is any material known in the art that can be blended with the polymer (e.g., the polymer of the drug release matrix) to increase its mechanical flexibility. Plasticizers may also affect the drugs' incorporation into the polymer of the drug release matrix and release kinetics. Any biocompatible plasticizer known in the art may be used. Examples of plasticizers include compounds of the pluronic/poloxamer non-ionic surfactant family and lipids. Particular plasticizers that may be used include Span 20, Tween 20, propylene glycol, sorbitan monolaurate and propylene glycol, and combinations thereof. In various embodiments, the contact lens device includes 1-90% by weight of a plasticizer, e.g., 5-50% by weight, 10-30% by weight, or 15-25% by weight. In one, non-limiting embodiment, the drug release matrix of a device includes, by weight, 30% drug, 50% polymer, and 20% plasticizer.

The device may include one or more antibacterial agents for controlled release. Non-limiting examples of suitable antibacterial agents include bacitracin, chloramphenicol, ciprofloxacin, erythromycin, moxifloxacin, gatifloxacin, gentamicin, levofloxacin, sulfacetamide, polymyxin B, vancomycin, tobramycin, or a combination thereof. Other antibacterial agents may be incorporated into and released from the device.

Antiviral agents include, but are not limited to, trifluridine, vidarabine, acyclovir, valacyclovir, famciclovir, foscarnet, ganciclovir, formivirsen, and cidofovir.

Antifungal agents include, but are not limited to, amphotericin B, natamycin, fluconazole, itraconazole, ketoconazole, and miconazole.

Antiprotozoal agents include, but are not limited to, polymyxin B, neomycin, clotrimazole, miconazole, ketoconazole, propamidine, polyhexamethylene biguanide, chlorhexidine, itraconazole.

Anesthetic agents include, but are not limited to, an aminoamide, an aminoester, or a mixture thereof. Combinations of different aminoamides or combinations of different aminoesters are envisioned. Representative examples of possible aminoamides include lidocaine, prilocalne, mepivacaine, and ropivacaine. Representative examples of possible aminoesters include benzocaine, procaine, proparacaine, and tetracaine.

Autonomic agents include, but are not limited to, acetylcholine, carbachol, pilocarpine, physostigmine, echothiophate, atropine, scopolamine, homatropine, cyclopentolate, tropicamide, dipivefrin, epinephrine, phenylephrine, apraclonidine, brimonidine, cocaine, hydroxyamphetamine, naphazoline, tatrahydrozoline, dapiprazole, betaxolol, carteolol, levobunolol, metipranolol, and timolol.

Anti-inflammatory agents include, but are not limited to, any known non-steroidal anti-inflammatory agent, and any known steroidal anti-inflammatory agent. Non-limiting examples include glucocorticoids (e.g., dexamethasone, prednisolone, fluorometholone, loteprednol, medrysone, and rimexolone) and NSAIDS (e.g., diclofenac, flurbiprofen, ketorolac, bromfenac, and nepafenac).

Antihistamines include, but are not limited to, pheniramine, antazoline, naphazoline, emedastine, levocabastine, and cromolyn.

Mast-cell stabilizers include, but are not limited to, lodoxamide, pemirolast, nedocromil, olopatadine, ketotifen, azelastine, and epinastine.

Antimicrobial agents include antibiotics (e.g. antibacterial), antiviral agents, antifungal agents, and anti-protozoan agents.

Antineoplastic agents include, but are not limited to, those which are suitable for treating tumors of the eye and its adnexa including cancer chemotherapeutic agents, a variety of which are well known in the art.

The drug release matrix and/or lens matrix described herein may be used in “pharmaceutical compositions” or “pharmaceutically acceptable” compositions, which comprise a therapeutically effective amount of an active agent associated with one or more of the drug release matrix and/or lens matrix described herein, formulated together with one or more pharmaceutically acceptable carriers, additives, and/or diluents. The pharmaceutical compositions described herein may be useful for diagnosing, preventing, treating or managing a disease or bodily condition including eye conditions. In some cases, a composition includes a drug release matrix encapsulated in a hydrogel and placed proximate the eye.

The phrase “pharmaceutically-acceptable carrier” as used herein means a pharmaceutically-acceptable material, composition or vehicle, such as a liquid, gel or solid filler, diluent, excipient, or solvent encapsulating material, involved in carrying or transporting the subject compound, e.g., from a device or from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The amount of active agent which can be combined with a polymer or other carrier material to produce a single dosage form will vary depending upon the host being treated, and the particular mode of administration. The amount of active agent that can be combined with a polymer or other carrier material to produce a single dosage form will generally be that amount of the compound which produces a therapeutic effect. Generally, this amount will range from about 1% to about 99% of active ingredient, from about 5% to about 70%, or from about 10% to about 30%. It should be understood that ranges outside these ranges may be used as well.

Delivery systems suitable for use with devices described herein include time-release, delayed release, sustained release, or controlled release delivery systems. Many types of release delivery systems are available and known to those of ordinary skill in the art. Specific examples include, but are not limited to, erosional systems in which the composition is contained in a form within a matrix, or diffusional systems in which an active component controls the release rate. The compositions may be as, for example, particles (e.g., microparticles, drug release matrix, nanoparticles), hydrogels, polymeric reservoirs, or combinations thereof. In some embodiments, the system may allow sustained or controlled release of an active agent to occur, for example, through control of the diffusion or erosion/degradation rate of the formulation or particle. The devices described herein can also be combined (e.g., contained) with delivery devices such as syringes, catheters, tubes, and implantable devices.

When the devices described herein are administered as pharmaceuticals, to humans and animals, they can be given per se or as a pharmaceutical composition containing, for example, about 0.1% to about 99.5%, about 0.5% to about 90%, or the like, of drug release matrix in combination with a pharmaceutically acceptable carrier.

In some embodiments, the effective amount of any drug release described herein may be from about 1 ng/kg of body weight to about 10 mg/kg of body weight, and the frequency of administration may range from once a day to a once a month basis, to an as-needed basis. However, other dosage amounts and frequencies also may be used as the invention is not limited in this respect. A subject may be administered devices described herein in an amount effective to treat one or more diseases or bodily conditions described herein.

### Methods of Using Compounds of the Disclosure

The compounds of the present disclosure are useful in the treatment of diseases characterized by disruption of or damage to cGMP/PKG and/or cAMP/PKA signaling mediated pathways, e.g., as a result of increased expression of PDE1 or decreased expression of cGMP/PKG or cAMP/PKA activity due to inhibition or reduced levels of inducers of cyclic nucleotide synthesis, such as dopamine and nitric oxide (NO). It is believed that by inhibiting PDE1, for example, that this action could reverse or prevent the attenuation of cGMP/PKG or cAMP/PKA signaling (e.g., enhance cGMP or cAMP, respectively). Therefore, administration or use of a preferred PDE1 inhibitor as described herein, e.g., a PDE1 inhibitor as hereinbefore described, e.g., a Compound of Formula I, Ia, II, III, IV, is believed to provide a potential means of treatment for ophthalmic diseases, disorders or injuries (i.e., glaucoma or elevated intraocular pressure). Compound I has been shown to be neuroprotective and to promote synaptic regeneration (neurogenesis) after injury.

In various embodiments, the present disclosure provides for a method [Method 1] for treatment or prophylaxis of an ophthalmic disease, disorder or injury (e.g., glaucoma or elevated intraocular pressure), the method comprising administering a drug eluting contact lens (e.g., Device 1, et seq.) comprising a drug release matrix and a pharmaceutically effective amount of a PDE1 inhibitor, wherein the drug eluting contact lens is configured to release the PDE1 inhibitor over a sustained period of time (e.g., a compound according to Formula I, Ia, II, III, or IV) to a subject in need thereof. For example, the present disclosure provides for the following embodiments of Method 1:
1.1 Method 1, wherein the PDE1 inhibitor is a compound according to Formula I, Ia, II, III or IV.
1.2 Method 1 or 1.1, wherein the PDE1 inhibitor comprises (6aR,9aS)-5,6a,7,8,9,9a-hexahydro-5-methyl-3-(phenylamino)-2-((4-(6-fluoropyridin-2-yl)phenyl)methyl)-cyclopent[4,5]imidazo[1,2-a]pyrazolo[4,3-e]pyrimidin-4(2H)-one:

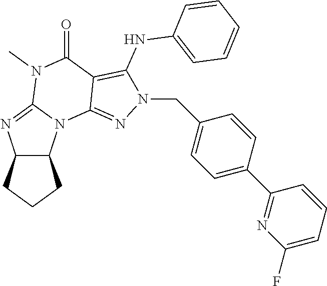 [SMILES: CN1C(=O)c2c(nn(Cc3ccc(-c4cccc(F)n4)cc3)c2Nc2ccccc2)N2C1=N[C@@H]1CCC[C@@H]12]

in free or pharmaceutically acceptable salt form.
1.3 Any foregoing Method, wherein the PDE1 inhibitor comprises 7,8-dihydro-2-(4-acetylbenzyl)-3-(4-fluorophenylamino)-5,7,7-trimethyl-[2H]-imidazo-[1,2-a]pyrazolo[4,3-e]pyrimidin-4(5H)-one:

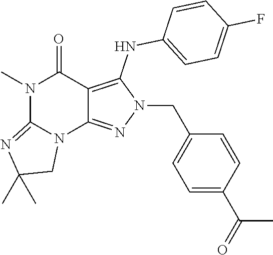 [SMILES: CC(=O)c1ccc(Cn2nc3c(c2Nc2ccc(F)cc2)C(=O)N(C)C2=NC(C)(C)CN23)cc1]

in free or pharmaceutically acceptable salt form.
1.4 Any foregoing Method, wherein the PDE1 inhibitor comprises 3-((4-fluorophenyl)amino)-5,7,7-trimethyl-2-((2-methylpyrimidin-5-yl)methyl)-7,8-dihydro-2H-imidazo[1,2-a]pyrazolo[4,3-e]pyrimidin-4(5H)-one

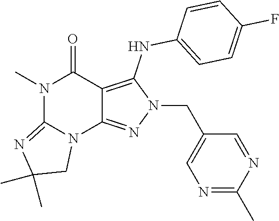 [SMILES: Cc1ncc(Cn2nc3c(c2Nc2ccc(F)cc2)C(=O)N(C)C2=NC(C)(C)CN23)cn1]

in free or pharmaceutically acceptable salt form.
1.5 Any foregoing Method, wherein the PDE1 inhibitor comprises (6aR,9aS)-5,6a,7,8,9,9a-hexahydro-5-methyl-3-(phenylamino)-2-((4-(pyridin-2-yl)phenyl)methyl)-cyclopent[4,5]imidazo[1,2-a]pyrazolo[4,3-e]pyrimidin-4(2H)-one:

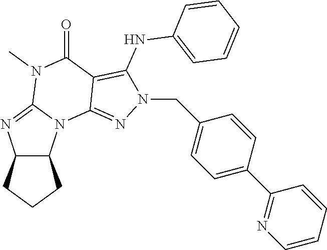 [SMILES: CN1C(=O)c2c(nn(Cc3ccc(-c4ccccn4)cc3)c2Nc2ccccc2)N2C1=N[C@@H]1CCC[C@@H]12]

in free or pharmaceutically acceptable salt form.
1.6 Any foregoing Method, wherein the drug release matrix comprises a biodegradable polymer, a nondegradable polymer, a hydrogel or a combination thereof.
1.7 Any foregoing Method, wherein the drug release matrix comprises or consists of a biodegradable polymer.
1.8 Any foregoing Method, wherein the drug release matrix comprises a degradable polymer selected from the group consisting of poly(lactic-co-glycolic) acid (“PLGA”), polylactide, polyglycolide, polycaprolactone, or other polyesters, poly(orthoesters), poly(aminoesters), polyanhydrides, polyorganophosphazenes, or combinations thereof.
1.9 Any foregoing Method, wherein the drug release matrix comprises or consists of a non-degradable polymer.
1.10 Any foregoing Method, wherein the drug release matrix comprises non-degradable polymer selected from the group consisting of ethyl cellulose, poly(butyl acrylate), poly(urethanes), silicone resins, nylon, ammonium polyacrylate, acrylamide copolymers, acrylate/acrylamide copolymers, acrylate/ammonium acrylate copolymers, acrylate/alkyl acrylate copolymers, acrylate/carbamate copolymers, acrylate/dimethylaminoethyl methacrylate copolymers, ammonium acrylate copolymers, styrene/acrylate copolymers, vinyl acetate/acrylate copolymers, aminomethylpropanol/acrylate/dimethylaminoethylmethacrylate copolymers, or combinations thereof.
1.11 Any foregoing Method, wherein the drug release matrix comprises or consists of a hydrogel.
1.12 Any foregoing Method, wherein the drug release matrix comprises a hydrogel selected from polyhydroxyethylmethacrylate (pHEMA), a silicone, agarose, alginate, chitosan, and hyaluronic acid.
1.13 Any preceding Method, wherein the drug release matrix comprises a biodegradable polymer, a nondegradable polymer or a hydrogel or a combination thereof in an amount of about 1-99% by weight of the total weight of the drug release matrix, about 50-90% by weight of the total weight of the drug release matrix, or about 60-80% by weight of the total weight of the drug release matrix.
1.14 Any foregoing Method, wherein the drug release matrix is in the form of a film configured to fit over the cornea of a subject in need thereof.
1.15 Any preceding Method, wherein the drug release matrix is loaded with a concentration of the drug toward perimeter of the device, such that the drug release matrix substantially does not release the drug from a portion of the device aligning over a subject's pupil.
1.16 Any foregoing Method, wherein the drug-eluting contact lens further comprises a lens matrix that a) forms a layer abutting the drug release matrix or b) at least partially encapsulates the drug release matrix.
1.17 Any foregoing Method, wherein the drug eluting contact lens comprises a first layer comprising the drug release matrix and a second layer comprising a non-eluting material, such that when in use the first layer faces toward the surface of the eye.
1.18 The preceding Method, wherein the non-eluting material is a non-degradable polymer.
1.19 The two preceding Method, wherein the non-eluting material comprises the lens matrix.
1.20 Any preceding Method, wherein the drug-eluting contact lens comprises at least one additional active agent.
1.21 The preceding Method wherein, the additional active agent is an intraocular pressure-lowering agent, growth factors; angiogenic agents; anti-inflammatory agents; anti-infective agents such as antibacterial agents, antiviral agents, antifungal agents, and agents that inhibit protozoan infections; antineoplastic agents; anesthetics; anti-cancer compositions; autonomic agents; steroids (e.g., corticosteroids); non-steroidal anti-inflammatory drugs (NSAIDs); antihistamines; mast-cell stabilizers; immunosuppressive agents; antimitotic agents; or combinations thereof.
1.22 Any preceding Method, wherein the additional active agent is an intraocular pressure-lowering agent.
1.23 The preceding Method, wherein the intraocular pressure-lowering agent is an adrenergic agonist, a beta-adrenergic antagonist, a prostaglandin or prostaglandin analog or a muscarinic analog, or an agent that raises cyclic nucleotides, a sympathomimetic, a miotic agent, a carbonic anhydrase inhibitor, a prostanoid, physostigmine, bimatoprost, brimonidine tartrate, or brimonidine tartrate/timolol maleate, or a combination thereof.
1.24 The preceding Method, wherein the prostaglandin is travoprost, latanoprost, bimatroprost, unoprostone, and unoprostone isopropyl.
1.25 The preceding Method, wherein the alpha adrenergic agonist is brimonidine, apraclonidine or dipivefrin.
1.26 The preceding Method, wherein the muscarinic agonist is pilocarpine.
1.27 Any preceding Method, wherein the device contains 0.1 μg to 100 mg of the PDE1 inhibitor, 1 μg to 100 mg of the PDE1 inhibitor, 10 μg to 100 mg of the PDE1 inhibitor, 50 μg to 100 mg of the PDE1 inhibitor, 100 μg to 100 mg of the PDE1 inhibitor, 500 μg to 50 mg of the PDE1 inhibitor, 1 mg to 50 mg of the PDE1 inhibitor, 5 mg to 50 mg of the PDE1 inhibitor, 10 mg to 40 mg of the PDE1 inhibitor, 20 mg to 35 mg of the PDE1 inhibitor, e.g., 10 mg to 30 mg of the PDE1 inhibitor.
1.28 Any preceding Method, wherein the PDE1 inhibitor is released at a constant rate (e.g., a zero order release profile) or at a higher initial concentration that tapers over time (e.g., a first order release profile) over a sustained period of time.
1.29 Any preceding Method, wherein the PDE1 inhibitor is released at a constant rate (e.g., a zero order release profile) over a sustained period of time.
1.30 Any preceding Method, wherein the PDE1 inhibitor is released at a rate of rate of 0.001 μg per hour, 0.005 μg per hour, 0.01 μg per hour, 0.05 μg per hour, 0.1 μg per hour, 0.5 μg per hour, 1 μg per hour, 5 μg per hour, 10 μg per hour, 20 μg per hour, 50 μg per hour, 100 μg per hour, 500 μg per hour, 1 mg per hour, or 5 mg per hour.
1.31 Any preceding Method, wherein the contact lens provides sustained release of the PDE1 inhibitor for at least 1 hour, at least 4 hours, at least 8 hours, at least 16 hours, at least 24 hours, at least 72 hours, at least 168 hours, at least 336 hours, at least 720 hours, at least 1440 hours, at least 2160 hours, at least 2400 hours, at least 2880 hours, at least 3600 hours, at least 4800 hours, at least 7200 hours, at least 9600 hours, or even longer.
1.32 Any preceding Method, wherein the contact lens provides sustained release of the PDE1 inhibitor for 24 hours, 2 weeks, one month or three months.
1.33 Any preceding Method, further comprising at least one plasticizer.
1.34 The preceding Method, wherein the plasticizer is a pluronic/poloxamer non-ionic surfactant or a lipid.
1.35 The preceding Method, wherein the plasticizer is Span 20, Tween 20, propylene glycol, sorbitan monolaurate and propylene glycol, and combinations thereof.
1.36 Any preceding Method, wherein the PDE1 inhibitor is present in an amount of about 1% to about 99% by weight of the drug release matrix, from about 5% to about 70% by weight, or from about 10% to about 30% by weight.
1.37 Any preceding Method, wherein the disease, disorder or injury is optic nerve injury or trauma, retinal injury or trauma, blindness consequent to diabetes, optic neuritis (e.g., optic neuritis consequent to infection or optic neuritis consequent to an autoimmune disorder such as multiple sclerosis or systemic lupus erythematosus), cancers, infections selected from bacterial and viral, glaucoma or elevated intraocular pressure.
1.38 The preceding Method, wherein the disease, disorder or injury is optic nerve injury or trauma.
1.39 Method 1.36, wherein the disease, disorder or injury is retinal injury or trauma.
1.40 Method 1.36, wherein the disease, disorder or injury is blindness consequent to diabetes.
1.41 Method 1.36, wherein the disease, disorder or injury is optic neuritis.
1.42 The preceding Method, wherein optic neuritis is consequent to infection or an autoimmune disorder such as multiple sclerosis or systemic lupus erythematosus.
1.43 Method 1.36, wherein the disease, disorder or injury is glaucoma or elevated intraocular pressure.
1.44 Method 1.36, wherein the disease is glaucoma.
1.45 Any preceding Method, wherein the contact lens is removable after being placed in contact with a subject's eye.
1.46 Any preceding Method, wherein the contact lens is disposable.
1.47 Any preceding Method, wherein the contact lens is left on the subject's eye until the entire drug load of PDE1 inhibitor has been released.
1.48 Any preceding Method, leaving the contact lens on the subject's eye until the contact lens fully degrades.

The disclosure further provides a PDE1 inhibitor for use in a method for the treatment or prophylaxis of an ophthalmic disease, disorder or injury (e.g., glaucoma or intraocular pressure) in a subject in need thereof, e.g., for use in any of Methods 1, et seq.

The disclosure further provides the use of a PDE1 inhibitor in the manufacture of a medicament for the treatment or prophylaxis of an ophthalmic disease, disorder or injury (e.g., glaucoma or intraocular pressure), e.g., a medicament for use in any of Methods 1, et seq.

Pharmaceutical compositions comprising Compounds of the Disclosure may be prepared using conventional diluents or excipients and techniques known in the galenic art. Thus, oral dosage forms may include tablets, capsules, solutions, suspensions and the like.

### EXAMPLES

### Example 1: Measurement of PDEIB Inhibition In Vitro Using IMAP Phosphodiesterase Assay Kit

Phosphodiesterase I B (PDEIB) is a calcium/calmodulin dependent phosphodiesterase enzyme that converts cyclic guanosine monophosphate (cGMP) to 5′-guanosine monophosphate (5′-GMP). PDEIB can also convert a modified cGMP substrate, such as the fluorescent molecule cGMP-fluorescein, to the corresponding GMP-fluorescein. The generation of GMP-fluorescein from cGMP-fluorescein can be quantitated, using, for example, the IMAP (Molecular Devices, Sunnyvale, Calif.) immobilized-metal affinity particle reagent.

Briefly, the IMAP reagent binds with high affinity to the free 5′-phosphate that is found in GMP-fluorescein and not in cGMP-fluorescein. The resulting GMPfluorescein-IMAP complex is large relative to cGMP-5 fluorescein. Small fluorophores that are bound up in a large, slowly tumbling, complex can be distinguished from unbound fluorophores, because the photons emitted as they fluoresce retain the same polarity as the photons used to excite the fluorescence.

In the phosphodiesterase assay, cGMP-fluorescein, which cannot be bound to IMAP, and therefore retains little fluorescence polarization, is converted to GMPfluorescein, which, when bound to IMAP, yields a large increase in fluorescence polarization (Amp). Inhibition of phosphodiesterase, therefore, is detected as a decrease in Amp. Enzyme assay

Materials: All chemicals are available from Sigma-Aldrich (St. Louis, Mo.) except for IMAP reagents (reaction buffer, binding buffer, FL-GMP and IMAP beads), which are available from Molecular Devices (Sunnyvale, Calif.).

Assay: The following phosphodiesterase enzymes may be used: 3′,5′-cyclic-nucleotide specific bovine brain phosphodiesterase (Sigma, St. Louis, Mo.) (predominantly PDEIB) and recombinant full length human PDE1A and PDE1B (r-hPDE1 A and r-hPDE1B respectively) which may be produced e.g., in HEK or SF9 cells by one skilled in the art. The PDE1 enzyme is reconstituted with 50% glycerol to 2.5 U/ml. One unit of enzyme will hydrolyze 1.0 m of 3′,5′-cAMP to 5′-AMP per min at pH 7.5 at 300° C. One part enzyme is added to 1999 parts reaction buffer (30 μM CaCl 2, 10 U/ml of calmodulin (Sigma P2277), 10 mM Tris-HCl pH 7.2, 10 mM MgCl 2, 0.1% BSA, 0.05% NaN 3) to yield a final concentration of 1.25 mU/ml. 99 μM of diluted enzyme solution is added into each well in a flat bottom 96-well polystyrene plate to which 1 μM of test compound dissolved in 100% DMSO is added. The compounds are mixed and pre-incubated with the enzyme for 10 min at room temperature.

The FL-GMP conversion reaction is initiated by combining 4 parts enzyme and inhibitor mix with 1 part substrate solution (0.225 μM) in a 384-well microtiter plate. The reaction is incubated in dark at room temperature for 15 min. The reaction is halted by addition of 60 μM of binding reagent (1:400 dilution of IMAP beads in binding buffer supplemented with 1:1800 dilution of antifoam) to each well of the 384-well plate. The plate is incubated at room temperature for 1 hour to allow IMAP binding to proceed to completion, and then placed in an Envision multimode microplate reader (PerkinElmer, Shelton, Conn.) to measure the fluorescence polarization (Amp).

A decrease in GMP concentration, measured as decreased Amp, is indicative of inhibition of PDE activity. IC50 values are determined by measuring enzyme activity in the presence of 8 to 16 concentrations of compound ranging from 0.0037 nM to 80,000 nM and then plotting drug concentration versus AmP, which allows IC50 values to be estimated using nonlinear regression software (XLFit; IDBS, Cambridge, Mass.).

### Example 2: Evaluation of the Present PDE1 Inhibitors' in Ocular Tissue Following a Topical Administration

The goal of this study was to determine the presence/absence of Compound 1 in male hypertensive New Zealand White Rabbit plasma and ocular tissue following a topical administration.

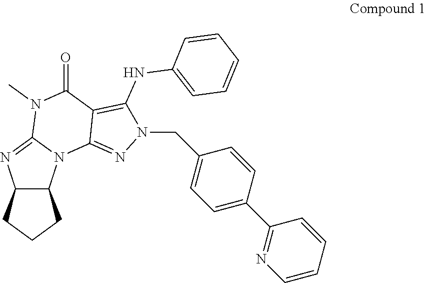 [SMILES: CN1C(=O)c2c(nn(Cc3ccc(-c4ccccn4)cc3)c2Nc2ccccc2)N2C1=N[C@@H]1CCC[C@@H]12]

For the study, Compound 1 was formulated with 5% (2-Hydroxypropyl)-β-cyclodextrin in phosphate-buffered saline pH 7.4 at 60 μg/eye. Plasma, Cornea, Conjunctiva, Aqueous Humor (AH), and Iris Ciliary Body (ICB) samples were collected for analysis of the concentrations of Compound 1 using LC-MS/MS methods.

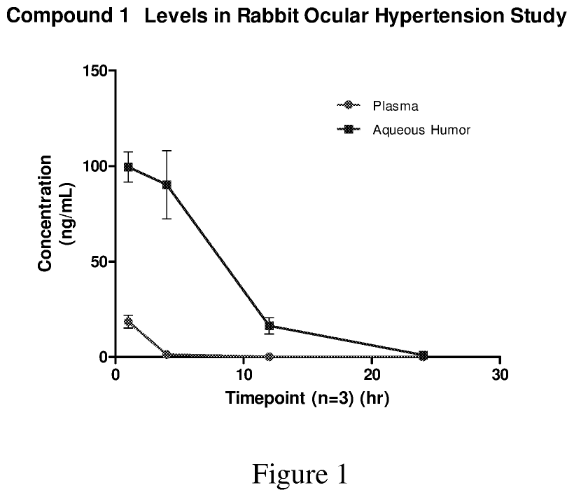

Compound 1 was present in plasma of the hypertensive rabbits 1 hour after topical administration of compound, signaling effective delivery of compound to the eye. The observed AUC₀₋₂₄ₕᵣ of Compound 1 in the aqueous humor was calculated to be 865.5 ng-hr/mL versus 46.3 ng-hr/mL in the plasma. The results are summarized in FIG. 1, which shows rapid availability of compound 1 in ocular tissue upon topical administration.

1. A drug eluting contact lens comprising a pharmaceutically effective amount of a PDE1 inhibitor encapsulated in a drug release matrix, wherein the drug eluting contact lens is configured to release the PDE1 inhibitor over a sustained period of time.

2. The drug eluting contact lens of claim 1, wherein the PDE1 inhibitor is a compound according to Formula I: 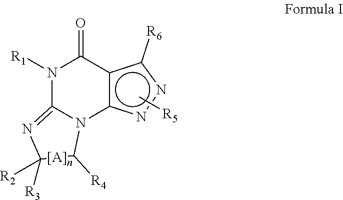
wherein
(i) R₁ is H or C₁₋₄ alkyl;
(ii) R₄ is H or C₁₋₄ alkyl and R₂ and R₃ are, independently, H or C₁₋₄ alkyl, aryl, heteroaryl, (optionally hetero)arylalkoxy, or (optionally hetero)arylalkyl; or
R₂ is H and R₃ and R₄ together form a di-, tri- or tetramethylene bridge;
(iii) R₅ is a substituted heteroarylalkyl;
or R₅ is attached to one of the nitrogens on the pyrazolo portion of Formula I and is a moiety of Formula A 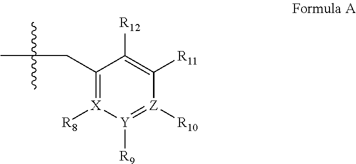
wherein X, Y and Z are, independently, N or C, and R₈, R₉, R₁₁ and R₁₂ are independently H or halogen, and R₁₀ is halogen, alkyl, cycloalkyl, haloalkyl, aryl, heteroaryl, diazolyl, triazolyl, tetrazolyl, arylcarbonyl, alkylsulfonyl, heteroarylcarbonyl, or alkoxycarbonyl; provided that when X, Y, or Z is nitrogen, R₈, R₉, or R₁₀, respectively, is not present; and
(iv) R₆ is H, alkyl, aryl, heteroaryl, arylalkyl, arylamino, heteroarylamino, N,N-dialkylamino, N,N-diarylamino, or N-aryl-N-(arylakyl)amino; and
(v) n=0 or 1;
(vi) when n=1, A is —C(R₁₃R₁₄)—
wherein R₁₃ and R₁₄, are, independently, H or C₁₋₄ alkyl, aryl, heteroaryl, (optionally hetero)arylalkoxy or (optionally hetero)arylalkyl;
in free, salt or prodrug form, including its enantiomers, diastereoisomers and racemates.

3. The drug eluting contact lens of claim 1, wherein the PDE1 inhibitor is a compound according to Formula Ia: 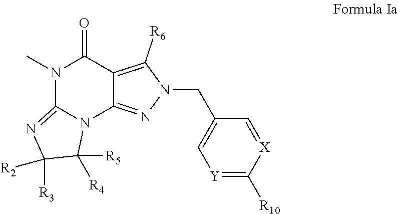
wherein
(i) R₂ and R₅ are independently H or hydroxy and R₃ and R₄ together form a tri- or tetra-methylene bridge; or R₂ and R₃ are each methyl and R₄ and R₅ are each H; or R₂, R₄ and R₅ are H and R₃ is isopropyl;
(ii) R₆ is (optionally halo-substituted) phenylamino, (optionally halo-substituted) benzylamino, C₁₋₄alkyl, or C₁₋₄alkyl sulfide; for example, phenylamino or 4-fluorophenylamino;
(iii) R₁₀ is C₁₋₄alkyl, methylcarbonyl, hydroxyethyl, carboxylic acid, sulfonamide, optionally halo- or hydroxy-substituted phenyl, optionally halo- or hydroxy-substituted pyridyl, or thiadiazolyl; and
(iv) X and Y are independently C or N,
in free, pharmaceutically acceptable salt or prodrug form, including its enantiomers, diastereoisomers and racemates.

4. The drug eluting contact lens of claim 1, wherein the PDE1 inhibitor is a compound of Formula II: 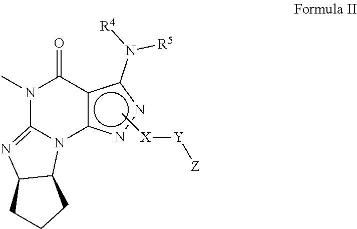
(i) X is C₁₋₆alkylene;
(ii) Y is a single bond, alkynylene, arylene or heteroarylene;
(iii) Z is H, aryl, heteroaryl, halo, haloC₁₋₆alkyl, C(O)—R¹, N(R²)(R³), or C₃₋₇cycloalkyl optionally containing at least one atom selected from a group consisting of N or O;
(iv) R¹ is C₁₋₆alkyl, haloC₁₋₆alkyl, OH or OC₁₋₆alkyl;
(v) R² and R³ are independently H or C₁₋₆alkyl;
(vi) R⁴ and R⁵ are independently H, C₁₋₆alky or aryl optionally substituted with one or more halo, hydroxy, or C₁₋₆alkoxy;
(vii) wherein X, Y and Z are independently and optionally substituted with one or more halo, C₁₋₆alkyl, haloC₁₋₆alkyl, haloC₁₋₆alkyl or C₁₋₆-alkyl, or Z is aryl, e.g., phenyl, substituted with one or more halo,
in free, pharmaceutically acceptable salt or prodrug form, including its enantiomers, diastereoisomers and racemates.

5. The drug eluting contact lens of claim 1, wherein the PDE1 inhibitor is a compound of Formula III: 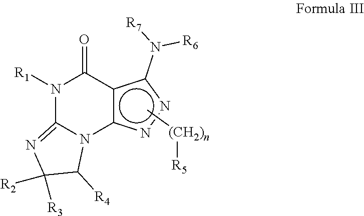
wherein
(i) R₁ is H or C₁₋₄ alkyl;
(ii) R₂ and R₃ are independently H or C₁₋₆ alkyl;
(iii) R₄ is H or C₁₋₄ alkyl;
(iv) R₅ is aryl optionally substituted with one or more groups independently selected from —C(═O)—C₁₋₆ alkyl and C₁₋₆-hydroxyalkyl;
(v) R₆ and R₇ are independently H or aryl optionally substituted with one or more groups independently selected from C₁₋₆ alkyl and halogen, for example unsubstituted phenyl or phenyl substituted with one or more halogen or phenyl substituted with one or more C₁₋₆ alkyl and one or more halogen or phenyl substituted with one C₁₋₆ alkyl and one halogen; and
(vi) n is 1, 2, 3, or 4,
in free, pharmaceutically acceptable salt or prodrug form, including its enantiomers, diastereoisomers and racemates.

6. The drug eluting contact lens of claim 1, wherein the PDE1 inhibitor is a compound of Formula IV: 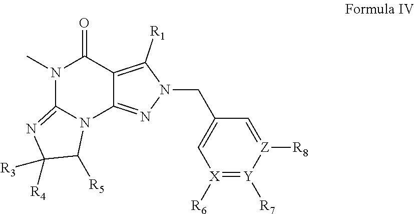
in free or salt form, wherein
(i) R₁ is C₁₋₄alkyl, or —NH(R₂), wherein R₂ is phenyl optionally substituted with halo;
(ii) X, Y and Z are, independently, N or C;
(iii) R₃, R₄ and R₅ are independently H or C₁₋₄alkyl; or R₃ is H and R₄ and R₅ together form a tri-methylene bridge,
(iv) R₆, R₇ and R₈ are independently:
H,
C₁₋₄alkyl,
pyrid-2-yl substituted with hydroxy, or
—S(O)₂—NH₂;
(v) Provided that when X, Y and/or Z are N, then R₆, R₇ and/or R₈, respectively, are not present; and when X, Y and Z are all C, then at least one of R₆, R₇ or R₈ is —S(O)₂—NH₂ or pyrid-2-yl substituted with hydroxy,
in free, pharmaceutically acceptable salt or prodrug form, including its enantiomers, diastereoisomers and racemates.

7. The drug eluting contact lens of claim 1, wherein the PDE1 inhibitor is: 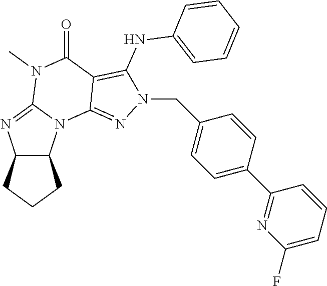 [SMILES: CN1C(=O)c2c(nn(Cc3ccc(-c4cccc(F)n4)cc3)c2Nc2ccccc2)N2C1=N[C@@H]1CCC[C@@H]12]
in free or pharmaceutically acceptable salt form.

8. The drug eluting contact lens of claim 1, wherein the PDE1 inhibitor is: 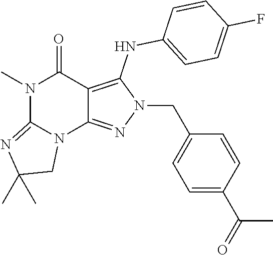 [SMILES: CC(=O)c1ccc(Cn2nc3c(c2Nc2ccc(F)cc2)C(=O)N(C)C2=NC(C)(C)CN23)cc1]
in free or pharmaceutically acceptable salt form.

9. The drug eluting contact lens of claim 1, wherein the PDE1 inhibitor is: 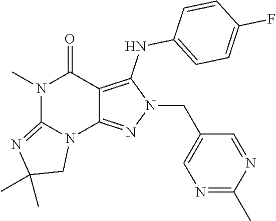 [SMILES: Cc1ncc(Cn2nc3c(c2Nc2ccc(F)cc2)C(=O)N(C)C2=NC(C)(C)CN23)cn1]
in free or pharmaceutically acceptable salt form.

10. The drug eluting contact lens of claim 1, wherein the PDE1 inhibitor is: 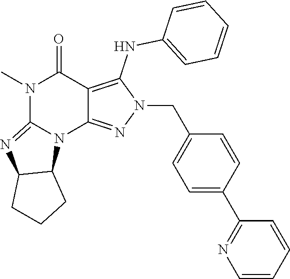 [SMILES: CN1C(=O)c2c(nn(Cc3ccc(-c4ccccn4)cc3)c2Nc2ccccc2)N2C1=N[C@@H]1CCC[C@@H]12]
in free or pharmaceutically acceptable salt form.

11. The drug eluting contact lens of claim 1, wherein the drug release matrix comprises a biodegradable polymer, a nondegradable polymer, a hydrogel or a combination thereof.

12. The drug eluting contact lens of claim 1, wherein the drug release matrix comprises or consists of a biodegradable polymer.

13. The drug eluting contact lens of claim 1, wherein the drug release matrix comprises a degradable polymer selected from the group consisting of poly(lactic-co-glycolic) acid (“PLGA”), polylactide, polyglycolide, polycaprolactone, or other polyesters, poly(orthoesters), poly(aminoesters), polyanhydrides, polyorganophosphazenes, or combinations thereof.

14. The drug eluting contact lens of claim 1, wherein the drug-eluting contact lens comprises at least one additional active agent.

15. The drug eluting contact lens of claim 1, the additional active agent is an intraocular pressure-lowering agent, growth factors; angiogenic agents; anti-inflammatory agents; anti-infective agents such as antibacterial agents, antiviral agents, antifungal agents, and agents that inhibit protozoan infections; antineoplastic agents; anesthetics; anti-cancer compositions; autonomic agents; steroids; non-steroidal anti-inflammatory drugs (NSAIDs); antihistamines; mast-cell stabilizers; immunosuppressive agents; antimitotic agents; or combinations thereof.

16. The drug eluting contact lens of claim 1, wherein the additional active agent is an intraocular pressure-lowering agent.

17. The drug eluting contact lens of claim 1, wherein the PDE1 inhibitor is released at a constant rate or at a higher initial concentration that tapers over time over a sustained period of time.

18. The drug eluting contact lens of claim 1, wherein the contact lens provides sustained release of the PDE1 inhibitor for 24 hours, 2 weeks, one month or three months.

19. The drug eluting contact lens of claim 1, wherein the contact lens is disposable.

20. A method for the treatment or prophylaxis of an ophthalmic disease, disorder or injury, the method comprising administering a drug eluting contact lens according to claim 1, wherein the drug eluting contact lens is configured to release the PDE1 inhibitor over a sustained period of time to a subject in need thereof.

21. The method of claim 20, wherein the ophthalmic disease, disorder or injury is optic nerve injury or trauma, retinal injury or trauma, blindness consequent to diabetes, glaucoma or elevated intraocular pressure.

22. The method of claim 20, wherein the ophthalmic disease, disorder or injury is glaucoma.
